(19) United States
(12) Patent Application Publication
(10) Pub. No.: US 2026/0356926 A1
(43) Pub. Date: Nov. 12, 2026

(54) NEUTRALIZING ANTIBODY AGAINST PULMONARY FIBROSIS, AND DRUG

(71) Applicant: Zhejiang University, Hangzhou City, Zhejiang Province (CN)

(72) Inventors: Ying Chi, Hangzhou City, Zhejiang Province (CN); Wangting Xu, Hangzhou City, Zhejiang Province (CN); Yuehai Ke, Hangzhou City, Zhejiang Province (CN); Xue Zhang, Hangzhou City, Zhejiang Province (CN); Caijuan Huan, Hangzhou City, Zhejiang Province (CN); Jianying Zhou, Hangzhou City, Zhejiang Province (CN)

(21) Appl. No.: 19/307,145

(22) Filed: Aug. 22, 2025

(30) Foreign Application Priority Data
Apr. 18, 2025 (CN) 202510487050.5

(51) Int. Cl. C07K 16/18; A61K 39/00; A61P 11/00

(52) U.S. Cl. CPC C07K 16/18; A61P 11/00; A61K 2039/505; C07K 2317/76

(57) ABSTRACT

A novel neutralizing antibody against pulmonary fibrosis and a drug. A neutralizing antibody targeting a binding site between Gremlin2 and TGF-β receptor 1 is designed on the basis of leading-edge artificial intelligence techniques such as Alphafold and de novo protein design, and an antibody is prepared by using genetic engineering techniques. Specifically, a gene fragment of an antibody of interest is inserted into a plasmid by in-vitro recombination, and subsequently the edited plasmid is transferred into a cell line, which is then expanded and cultured in vitro, and transcription and translation of the gene fragment are performed in-vitro, thereby preparing the antibody.

### CROSS-REFERENCE TO RELATED APPLICATION

This patent application claims the benefit and priority of Chinese Patent Application No. 202510487050.5 entitled “NOVEL NEUTRALIZING ANTIBODY AGAINST PULMONARY FIBROSIS, AND DRUG” filed with the China National Intellectual Property Administration on Apr. 18, 2025, the disclosure of which is incorporated by reference herein in its entirety as part of the present application.

### REFERENCE TO SEQUENCE LISTING

A computer readable XML file entitled “GWP20250601373-Sequence Listing”, that was created on Aug. 2, 2025, with a file size of about 11,069 bytes, contains the sequence listing for this application, has been filed with this application, and is hereby incorporated by reference in its entirety.

### TECHNICAL FIELD

The present disclosure belongs to the field of antibody drug preparation, and relates to a novel neutralizing antibody against pulmonary fibrosis and a drug.

### BACKGROUND

Idiopathic pulmonary fibrosis (IPF) is a typical type of fatal fabrotic disease.

The mechanism of sustained fibroblast activation may be critical to the rapid and irreversible progression of fibrosis, and the transforming growth factor (TGF-β) signaling has been identified as a critical mediator in the process of fibroblast-induced activation. Studies have shown that activated TGF-β1 (transforming growth factor β1) can bind to TGFBR2 (TGF-β Receptor 2, transforming growth factor β receptor 2) on the cell membrane, and then recruit intracellular TGFBR1 (TGF-β Receptor 1, transforming growth factor β receptor 1) to the membrane. This leads to the phosphorylation of TGFBR1, which enables signal transmission. Subsequently, this signaling cascade is terminated through ubiquitination-mediated degradation of membrane-bound TGFBR1. The present disclosure reveals that the binding of Gremlin2 to membrane-bound TGFBR1 stabilizes its signal transduction at the cell membrane, thereby leading to sustained activation of TGF-β signaling. Basic research has revealed that Gremlin2 is a ligand of TGFBR1. Through basic research and in-depth analysis by artificial intelligence (AI), a key binding epitope is identified. Monoclonal antibodies targeting the key epitope are designed by using an optimized AI algorithm.

Gremlin2, a protein of the TGF-β superfamily, is a potential target for anti-fibrotic therapy. Unlike the extensive regulation of TGF-β signaling in various cells, Gremlin2 is an important protein that specifically targets fibroblasts to regulate their activation. Clinical transformation of Gremlin2 neutralizing antibodies may bring new breakthroughs in the treatment of patients with pulmonary fibrosis.

### SUMMARY

An objective of the present disclosure is to provide a novel neutralizing antibody and therapeutic agent against pulmonary fibrosis, addressing the limitation of existing technologies.

In order to achieve the above objective, the present disclosure provides the following technical solutions: In a first aspect, provided is a neutralizing antibody against pulmonary fibrosis consisting of a heavy chain and a light chain, where the heavy chain includes a heavy chain variable region including a heavy chain CDR1 as set forth in SEQ ID NO. 1, a heavy chain CDR2 as set forth in SEQ ID NO. 2, and a heavy chain CDR3 as set forth in SEQ ID NO. 3; the light chain includes a light chain variable region including a light chain CDR1 as set forth in SEQ ID NO. 4, a light chain CDR2 as set forth in SEQ ID NO. 5, and a light chain CDR3 as set forth in SEQ ID NO. 6.

Further, an amino acid sequence of the heavy chain is as set forth in SEQ ID NO. 8, and an amino acid sequence of the light chain is as set forth in SEQ ID NO. 9.

Further, the neutralizing antibody against pulmonary fibrosis targets a specific epitope on Gremlin2, located at a binding site between Gremlin2 and TGFBR1.

In a second aspect, the present disclosure provides a pharmaceutical composition, including the neutralizing antibody against pulmonary fibrosis described above, intended for the treatment of pulmonary fibrosis diseases mediated by Gremlin2.

Further, the drug further includes: a pharmaceutically acceptable carrier.

The present disclosure has the following beneficial effects: According to the present disclosure, a neutralizing antibody targeting the Gremlin2-TGFBR1 binding site is designed by computer-based deep learning, which overcomes the problem of requiring for a large number of experimental animals in traditional neutralizing antibody preparation, and enables early screening for effective antibody sequences using computer-based analysis, such that the problem in conventional methods for preparing a large number of antibodies with unknown sequences and whose effects cannot be guaranteed is avoided, and the workload of researchers in subsequent cell and animal model validation is reduced. The present disclosure features process stability, safety and efficiency, providing a new approach to the clinical development of therapeutic antibodies. The Gremlin2 neutralizing antibody designed according to the present disclosure is suitable for use in subsequent clinical studies of pulmonary fibrosis treatment, demonstrating a significant anti-fibrotic efficacy. Moreover, the process is systematic, cost-effective, and easily scalable for widespread application.

### BRIEF DESCRIPTION OF THE DRAWINGS

FIG. 1 is a schematic diagram of AI simulation of the binding of Gremlin2 to TGFBR1;

FIGS. 2A-2C are a group of diagrams showing specific binding sequences between Gremlin2 and TGFBR1, in which FIG. 2A is a ribbon map (Cartoons) of the antigen-antibody docking site, FIG. 2B is an all-atom diagram of the antigen-antibody docking site, and FIG. 2C is a schematic diagram of the epitope of the antigen, where the green portion represents the epitope of the antigen, and the white portion represents the remaining portion of the antigen except for the epitope;

FIG. 3 is a schematic diagram of a Gremlin2 dimer and TGFBR1;

FIG. 4 is a schematic diagram of AI simulation of the Gremlin2-TGFBR1 binding site with a neutralizing antibody;

FIGS. 5A-5B are a group of schematic diagrams showing the production of the Gremlin2 neutralizing antibody, in which FIG. 5A is a diagram showing the SDS-PAGE (Sodium Dodecyl Sulfate-Polyacrylamide Gel Electrophoresis) detection results, FIG. 5B is a diagram showing the SEC-HPLC (Size Exclusion Chromatography-High Performance Liquid Chromatography) detection results;

FIGS. 6A-6C are a group of diagrams showing the LC-MS (Liquid Chromatography-Mass Spectrometry Detection) detection results, in which FIG. 6A is a diagram showing the LC-MS detection results of the undegraded antibody (Intact), FIG. 6B is a diagram showing the LC-MS detection results of the light chain (LC), and FIG. 6C is a diagram showing the LC-MS detection results of the heavy chain (HC);

FIGS. 7A-7C are a group of diagrams showing the detection of the inhibitory effect of the Gremlin2 neutralizing antibody on the activation of fibroblasts in vitro, in which FIG. 7A is a diagram showing the detection results of the binding ability of TGFBR1 to Gremlin2 under the interference of the neutralizing antibody at different concentrations, FIG. 7B is a schematic diagram of immunofluorescence staining for labeling the activation indicator α-SMA (red) of fibroblasts and DAPI (blue) for labeling nucleus, with a scale bar of 50 microns, and FIG. 7C is a statistical histogram of the α-SMA fluorescence signals, where * represents p<0.05, ** represents p<0.01, *** represents p<0.001, and ANOVA is used for multiple group comparisons;

FIGS. 8A-8D are a group of diagrams showing the anti-pulmonary fibrosis function of the Gremlin2 neutralizing antibody, in which FIG. 8A is a diagram showing the change in body weight of each group of mice, FIG. 8B is a diagram showing the lung tissue coefficient of each group of mice, and FIG. 8C is a diagram showing the content of hydroxyproline in the lung tissues of each group of mice, where NS represents p>0.05, ** represents p<0.01, *** represents p<0.001, and ANOVA is used for multiple group comparisons; FIG. 8D is a gross image of the lung tissues of each group of mice;

FIGS. 9A-9C are a group of schematic diagrams of Masson staining of the lung tissues of each group of mice, in which FIG. 9A is a schematic diagram of Masson staining of the PBS-IgG group, FIG. 9B is a schematic diagram of Masson staining of the BLM-IgG group, and FIG. 9C is a schematic diagram of Masson staining of the BLM-AIAB group, with a scale bar of 200 microns;

FIG. 10 is a diagram showing the results of labeling the activation level of fibroblasts (α-SMA) and the level of extracellular matrix (collagen 1 and fibronectin) in the lung tissues of the mice by the WB (Western Blot) method;

FIGS. 11A-11C are a group of diagrams showing the statistical results of labeling the lung tissues of the mice by the RT-QPCR (Real-time Quantitative Polymerase Chain Reaction) method, in which FIG. 11A is a diagram showing the statistical results of labeling Col1a1 (α1 chain of collagen 1) in the lung tissues of the mice by the RT-QPCR method, FIG. 11B is a diagram showing the statistical results of labeling Col1a2 (α2 chain of collagen 1) in the lung tissues of the mice by the RT-QPCR method, and FIG. 11C is a diagram showing the statistical results of labeling fibronectin in the lung tissues of the mice by the RT-QPCR method;

FIGS. 12A-12D are a group of diagrams showing the toxicity detection of the Gremlin2 neutralizing antibody, in which FIG. 12A is a gross image of the hearts of each group of mice, FIG. 12B is a gross image of the livers of each group of mice, FIG. 12C is a gross image of the spleens of each group of mice, and FIG. 12D is a gross image of the kidneys of each group of mice.

### DETAILED DESCRIPTION OF THE EMBODIMENTS

According to the present disclosure, antibody sequences for the binding site between Gremlin2 and TGFBR1 (or referred to as Gremlin2 target) are designed by using an optimization algorithm based on leading AI techniques such as Alphafold protein structure prediction and de novo protein design, and an antibody is prepared by using genetic engineering techniques. Specifically, a gene fragment of an antibody of interest is inserted into a plasmid by in-vitro recombination, and subsequently the edited plasmid is transferred into a cell line, which is then expanded and cultured in vitro, and transcription and translation of the gene fragment are performed in-vitro, thereby preparing the antibody. On the basis of this, a neutralizing antibody targeting the specific epitope on Gremlin2 (hereinafter referred to as Gremlin2 neutralizing antibody or AIAB) is developed. After treatment with the Gremlin2 neutralizing antibody at the cellular and animal levels, the activation of fibroblasts and the degree of bleomycin-induced pulmonary fibrosis in mice are significantly reduced. The subsequent humanization modification of the Gremlin2 neutralizing antibody may serve as a novel treatment method for patients with pulmonary fibrosis and potential patients.

Examples, features and aspects of the present disclosure will be described in detail below with reference to the drawings, which, however, are not intended to limit the present disclosure. Any embodiment extended on the basis of the examples of the present disclosure, and all other embodiments obtained by those of ordinary skill in the art without creative efforts, shall fall within the scope of protection of the present disclosure.

### Example 1: Design, Screening and Preparation of Gremlin2 Neutralizing Antibody

The Gremlin2 neutralizing antibodies obtained by screening in the present disclosure, which are mainly a class of biologically active recombinant proteins with high purity (about 1-3 mg/L), were dissolved in PBS after ultrafiltration membrane concentration and stored at −80 °C.
(1) Sequence design: Antibody sequences targeting a binding site between Gremlin2 and TGFBR1 (hereinafter referred to as Gremlin2 target) were designed by using an optimization algorithm based on leading AI algorithms such as Alphafold.

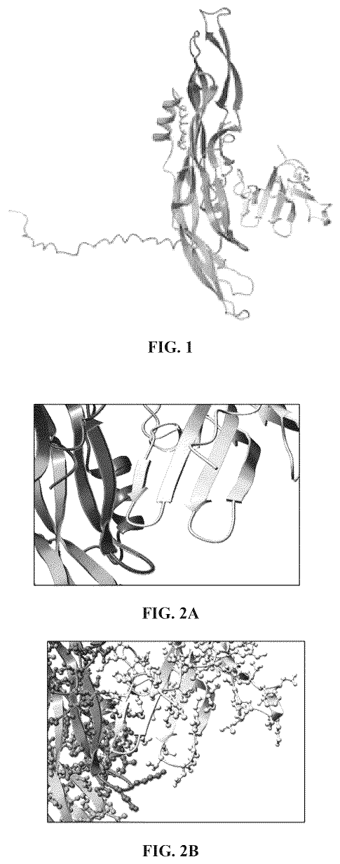

The binding of a Gremlin2 dimer (blue and pink) and an extracellular domain of TGFBR1 was predicted by simulating with an improved alpha-fold2 algorithm. As shown in FIG. 1, since the Gremlin2 dimer structure is composed of two monomers that are completely identical, the extracellular segment of TGFBR1 binds to two positions of the Gremlin2 dimer simultaneously, both of which are annotated by highlighting with green border in the figure.

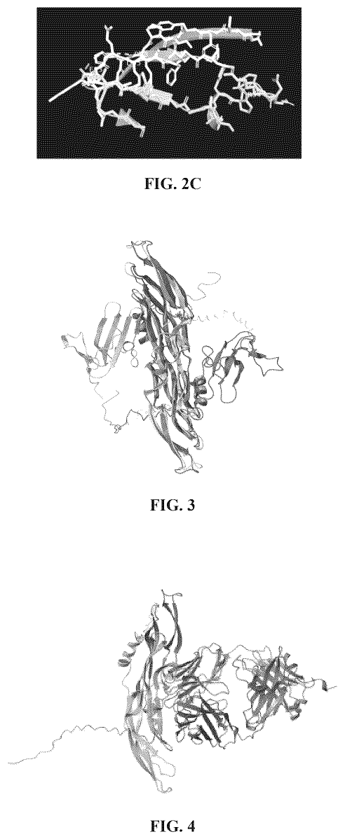

According to the sequence characteristics of the two proteins, binding sequences between the two (TGFBR1 and Gremlin2) are more clearly annotated by using computer simulation techniques in the present disclosure. As shown in FIG. 2, the main binding sequence on Gremlin2 to TGFBR1 is as set forth in SEQ ID NO. 7, and amino acids at positions 7, 12, 14, and 18 of the sequence are specific binding sites for direct binding to the TGFBR1 receptor.
(2) Sequence screening: The free energy of the binding between Gremlin2 and the designed sequences was measured and calculated by computer-based deep learning, and the stability of the Gremlin2 neutralizing antibodies was measured and calculated by methods such as conformational dynamics analysis, thereby screening for antibody sequences with high affinity for Gremlin2. For the finally selected antibody of the present disclosure, the amino acid sequence of the heavy chain is as follows:

(SEQ ID NO. 8)
EVQLVESGGGLVQPGGSLRLSCAASVSGDAPPAIGKYYMHWVRQAPGKGL
EWVGLIAGGEDGITIYDPKFQDRATISADNSKNTAYLQMNSLRAEDTAVY
YCARAPFGGAAYIEWGQGTLVTVSSASTKGPSVFPLAPSSKSTSGGTAAL
GCLVKDYFPEPVTVSWNSGALTSGVHTFPAVLQSSGLYSLSSVVTVPSSS
LGTQTYICNVNHKPSNTKVDKKVEPKSCDKTHT.

The amino acid sequence of the heavy chain variable region CDR1 is: VSGDAPPAIGKY (SEQ ID NO. 1); the amino acid sequence of CDR2 is: AGGEDGI (SEQ ID NO. 2); the amino acid sequence of CDR3 is: APFGGAAYIE (SEQ ID NO. 3).

The amino acid sequence of the light chain is as follows:

(SEQ ID NO. 9)
DIQMTQSPSSLSASVGDRVTITCSGGSGADIAWYQQKPGKAPKVLIYNGS
LPPGVPSRFSGSGSGTDYTLTISSLQPEDFATYYCAACTRKLTFGQGTKV
EIKRTVAAPSVFIFPPSDEQLKSGTASVVCLLNNFYPREAKVQWKVDNAL
QSGNSQESVTEQDSKDSTYSLSSTLTLSKADYEKHKVYACEVTHQGLSSP
VTKSFNRGEC.

The amino acid sequence of the light chain variable region CDR1 is:

(SEQ ID NO. 4)
SGGSGADIA;

the amino acid sequence of CDR2 is: NGSLPP (SEQ ID NO. 5); the amino acid sequence of CDR3 is:

(SEQ ID NO. 6)
AACTRKLT

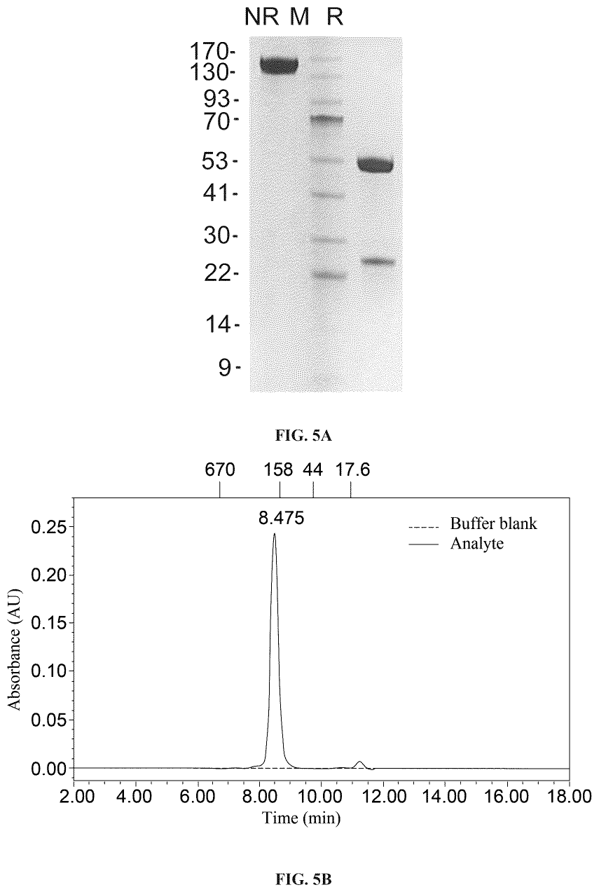

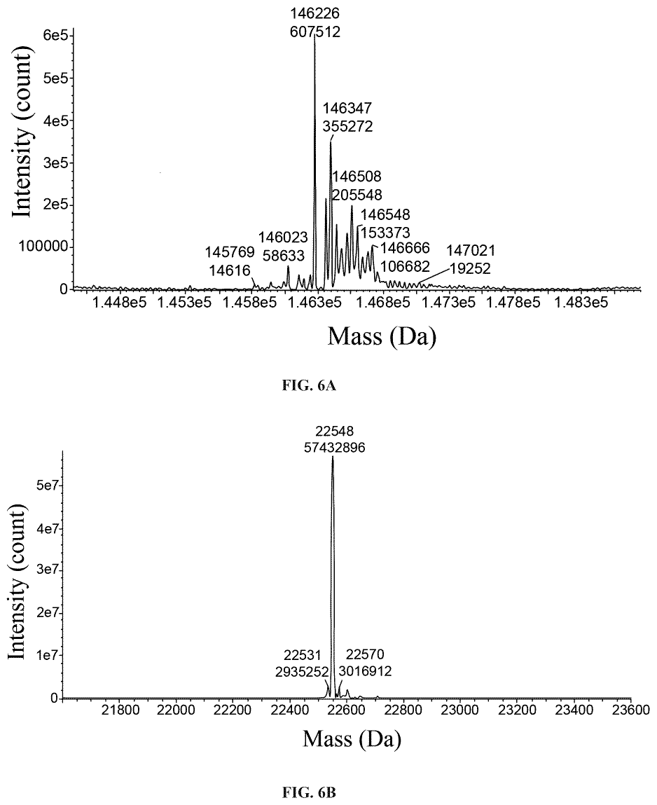

FIG. 3 is a schematic diagram of a Gremlin2 dimer and TGFBR1 (pink and blue represent the simulations of the binding of the TGFBR1 receptor to the two monomers of the Gremlin2 dimer, respectively), and FIG. 4 is a schematic diagram of AI simulation of the Gremlin2-TGFBR1 binding site with a neutralizing antibody. The binding free energy of the above sequences to the Gremlin2-TGFBR1 model was calculated. The smaller the value, the more stable the predicted binding of the sequences. The binding free energy in the original state is-7.13 kcal/mol, and the same binding sites on both sides have the same binding probability.
(3) Antibody synthesis: A gene fragment of an antibody of interest was inserted into a plasmid by using genetic engineering techniques, and subsequently the edited plasmid was transferred into a cell line; the edited cell line was proliferated and cultured in vitro; transcription and translation of a gene of interest were performed in vitro, thereby preparing a recombinant protein.
(4) Crude separation: The cells were collected, lysed at low temperature (4 °C) by using a lysis buffer containing a protease inhibitor, and disrupted by repeated freeze-thawing to release the recombinant protein.
(5) Concentration and purification: By using the ultrafiltration membrane concentration method, liquids such as water were filtered through a microporous cellulose membrane under high pressure, and the recombinant protein was retained on the membrane. After elution with sterile 1× PBS (phosphate buffered saline), the recombinant protein was determined for the concentration, aliquoted to maintain a stock concentration of 1-3 mg/mL, and stored at −80 °C.

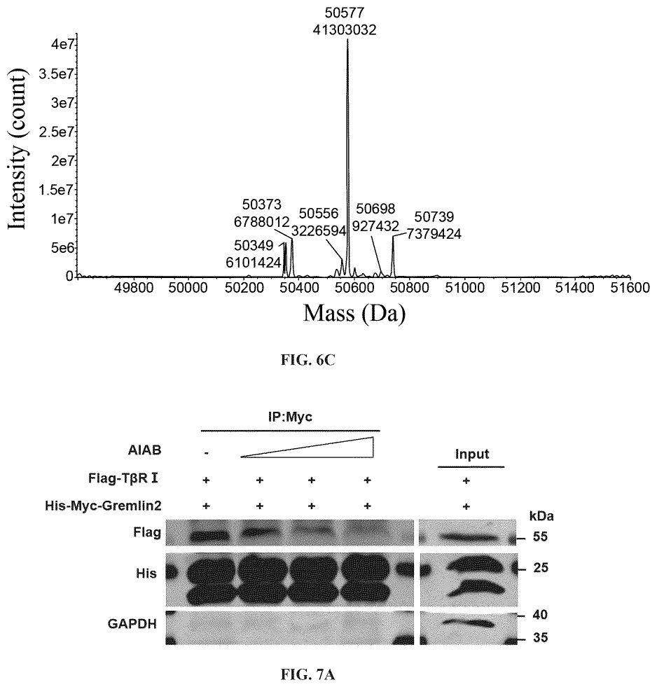

The protein size of the antibody was detected by SDS-PAGE, with the concentration of polyacrylamide gel ranging from 4-12% (w/v). As shown in FIG. 5A, lane M (Marker) represents a reference of molecular weight, NR (Non reduced) represents the size of the undegraded antibody (with light and heavy chains unseparated) (143.3 kDa), and lane R (Reduced) represents the size of the antibody with separated light and heavy chains (22-30 kDa for the light chain and about 53 kDa for the heavy chain). The electropherogram indicates that the antibody is a dimer.

Protein purity was detected by SEC-HPLC (SEC-300 chromatographic column) analysis. SEC-HPLC is mainly used to analyze and isolate substances of different molecular weights. As shown in FIG. 5B and Table 1, the antibody exhibited a single peak, suggesting that the antibody exists in a homogeneous form. The sample size was 20 μg, and the flow rate was 0.35 mL/min. ND denotes not detected.

TABLE 1
SEC-HPLC detection results

| Peak | Peak area (%) |
|---|---|
| High-molecular-weight substance | ND |
| Main peak | 100.0 |
| Low-molecular-weight substance | ND |

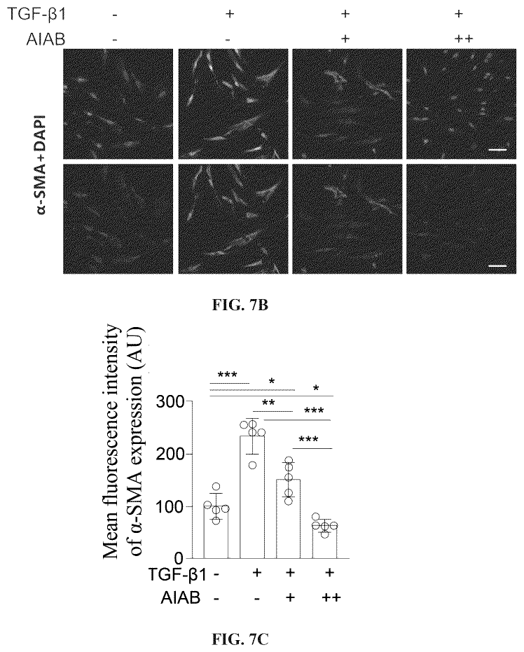

The LC-MS mass spectra, as shown in FIG. 6 and Table 2, showed that the undegraded antibody (Intact) primarily exhibited a molecular weight of about 143 kDa, with the heavy chain (HC) primarily exhibiting molecular weight of about 50 kDa and the light chain (LC) primarily exhibiting molecular weight of about 22 kDa. These results were consistent with the above results, further suggesting that the antibody exists in a dimeric form with good homogeneity.

TABLE 2
LC-MS detection results

| | Amino acid | Theoretical | Observed | Mass |
|---|---|---|---|---|
| Protein | mass (Da) | mass (Da) | mass (Da) | error (Da) |
| Undegraded | 143623.64 | 146226 | 146226 | 0 |
| antibody | | | | |
| Light chain | 22549.16 | 22549 | 22548 | −1 |
| Heavy chain | 49262.66 | 50580 | 50577 | −3 |

### Example 2: Validation of Inhibitory Effect of Gremlin2 Neutralizing Antibody on Activation of Fibroblasts In Vitro

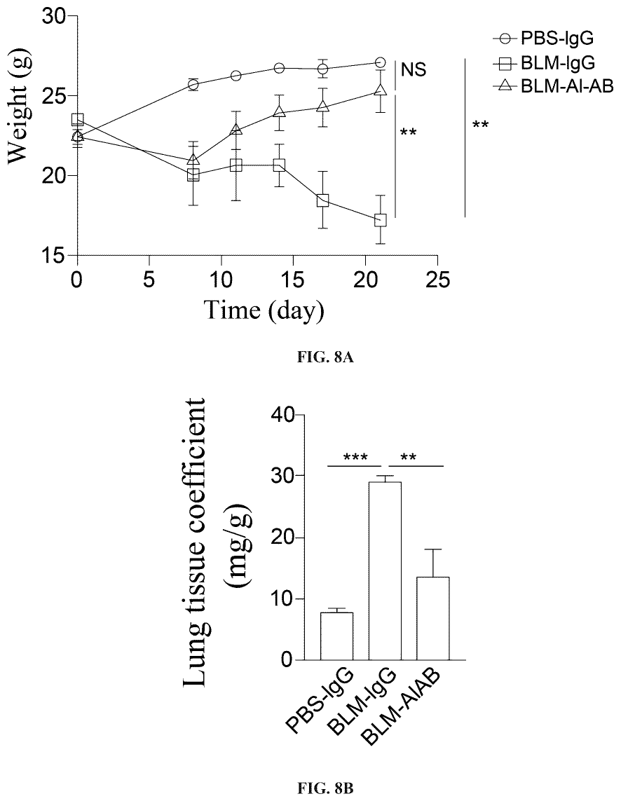

In 293T tool cells, TGFBR1 (TBRI) and Gremlin2 proteins were overexpressed with tags Flag and His-Myc, respectively. 72 hours after successful overexpression, 293T cells were lysed to obtain all proteins in the cells. Subsequently, the binding level of Gremlin2 to TGFBR1 was detected by Co-IP (Co-Immunoprecipitation) assay. By means of the conjugation of magnetic beads with a Myc antibody and on the basis of the principle of antigen-antibody binding, magnetic beads can be successfully conjugated with an Myc-tagged Gremlin2 protein. Different concentrations of Gremlin2 neutralizing antibody AIAB (concentrations: 0, 0.1, 0.2 and 0.3 μg/mL, respectively) were separately added to a protein lysate, and then the binding ability of TGFBR1 to Gremlin2 was detected. As shown in FIG. 7A, Input represents the presence of both TGFBR1 and Gremlin2 proteins in the original cell lysate; with Flag and His reflect the presence of TGFBR1 and Gremlin2, respectively; GAPDH (glyceraldehyde-3-phosphate dehydrogenase) serves as an internal reference protein; the IP group represents a gradual inhibition of the binding of TGFBR1 to Gremlin2 as antibody addition increases.

5 ng/ml of TGF-β1 stimulating factor was added to lung fibroblasts, followed by the addition of different concentrations of AI neutralizing antibody AIAB (concentrations: 0, 0.1 μg/mL and 0.2 μg/mL, respectively). Subsequently, the activation indicator α-SMA (red) of fibroblasts was labeled by immunofluorescence staining, and the nucleus was labeled with DAPI (blue). As shown in FIGS. 7B-7C, the mean fluorescence intensity of α-SMA decreased with increasing concentrations of the Gremlin2-neutralizing antibody, indicating that the Gremlin2 neutralizing antibody could inhibit the activation of fibroblasts in vitro.

### Example 3: Validation of Anti-Fibrotic Effect of Gremlin2 Neutralizing Antibody in Lung Fibrosis Animal Model

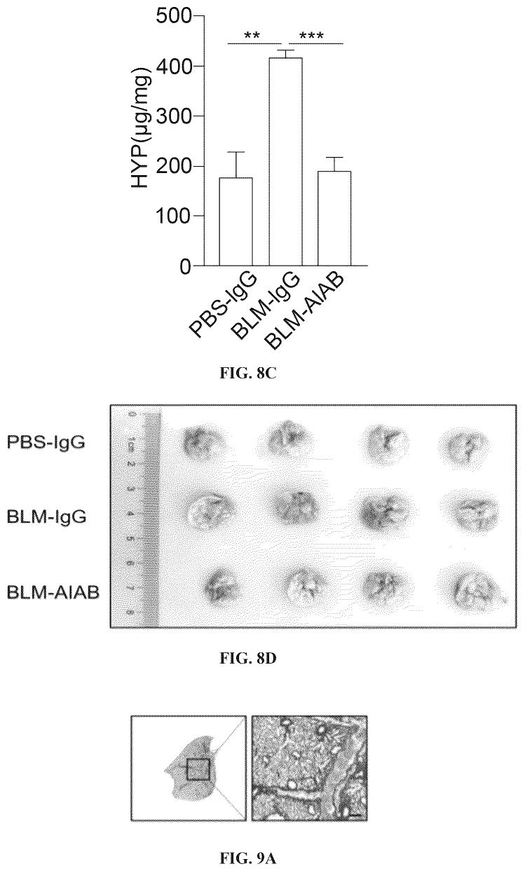

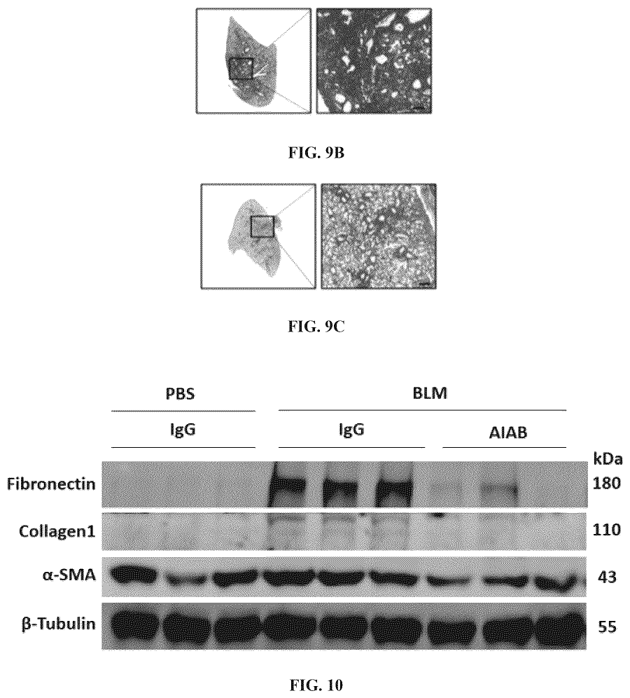

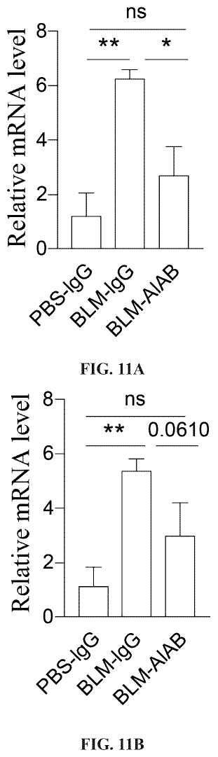

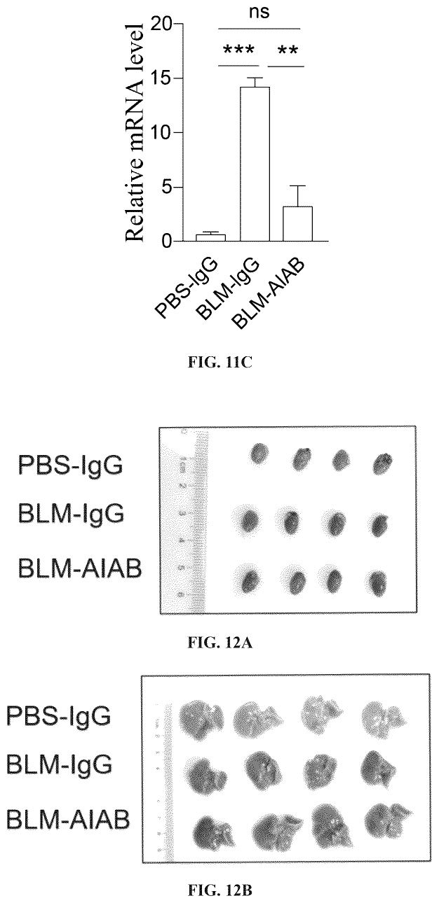

Male C57BL/6J mice aged 8 weeks were selected for the study. A pulmonary fibrosis mouse model was constructed by means of intratracheal instillation of PBS or bleomycin (BLM). 50 μg of Gremlin2 neutralizing antibodies AIABs or an equal volume of IgGs (immunoglobulin G) were injected intraperitoneally on days 8, 11, 14 and 17. The mice were divided into the following groups: a PBS-IgG group, a BLM-IgG group and a BLM-AIAB group.
(1) Changes in the body weight of the mice were recorded and plotted. As shown in FIG. 8A, no significant differences were observed between the PBS-IgG group and the BLM-AIAB group, while the body weight of mice in the BLM-IgG group was significantly lower than those in both the PBS-IgG group and the BLM-AIAB group. The diagram of the lung tissue coefficient of the mice is as shown in FIG. 8B, in which the lung tissue coefficient of the mice refers to the proportion of the weight of the lung tissues of the mice to the body weight of the mouse. The lung tissue coefficient of the mice in the BLM-AIAB group was significantly lower than that in the BLM-IgG group.
(2) Hydroxyproline (HYP) represents the severity of pulmonary fibrosis. The content of hydroxyproline in each milligram of wet lung tissue in mice was determined by using a hydroxyproline detection kit. As shown in FIG. 8C, the content of hydroxyproline in the lung tissues of the mice in the BLM-AIAB group was significantly lower than that in the BLM-IgG group.
(3) The lung tissue image of the mice is as shown in FIG. 8D. It could be observed that the damage area of the lung tissues of the mice increased after bleomycin modeling, while the damage area of the lung tissues decreased significantly after AIAB treatment. The lung tissues of the mice were subjected to Masson staining. As shown in FIG. 9, blue represents collagen deposition. It could be observed that the collagen deposition area in the lung tissues of the mice significantly expanded after bleomycin modeling, while the collagen deposition area in the lung tissues significantly shrank after AIAB treatment.
(4) The activation level of fibroblasts (α-SMA) and the level of extracellular matrix (collagen 1 and fibronectin) in the lung tissues of the mice were labeled by the WB method, with β-tubulin as the internal reference protein. As shown in FIG. 10, the activation level of fibroblasts in the lung tissues of the mice significantly increased after bleomycin modeling, as evidenced by a significant increase in the expression of α-SMA. Meanwhile, the expression of extracellular matrix also significantly increased, as evidenced by a significant increase in the expression of collagen 1 and fibronectin. In contrast, these fibrosis indicators were significantly inhibited after AIAB treatment.
(5) The level of extracellular matrix in the lung tissues of the mice was labeled by the RT-QPCR method. As shown in FIGS. 11A-11C, the expression of extracellular matrix in the lung tissues of the mice significantly increased after bleomycin modeling, as evidenced by a significant increase in the expression of collagen 1 (Col1a1 and Col1a2) and fibronectin. In contrast, these fibrosis indicators were significantly inhibited after AIAB treatment.

The above results indicate that the Gremlin2 neutralizing antibody AIAB has a better inhibitory effect on pulmonary fibrosis in mice.

### Example 4: Toxicity Detection of Gremlin2 Neutralizing Antibody

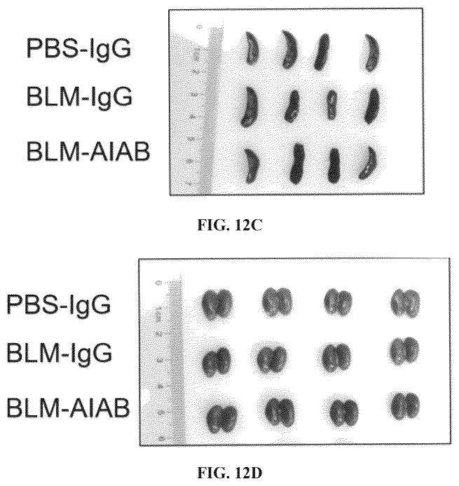

As shown in FIGS. 12A-12D, gross images of the heart, liver, spleen, and kidneys from the mice in the PBS-IgG group, BLM-IgG group, and BLM-AIAB group in Example 3 are presented. Overall characterization showed that intratracheal administration of bleomycin did not cause damage to organs other than the lung tissue, and exogenous application of the Gremlin2 neutralizing antibody also did not induce toxic injury to these organs.

It should be noted that the examples described above are merely preferred examples of the present disclosure, and are not intended to limit the present disclosure. For those skilled in the art, various changes and variations may be made to the present disclosure. Any modifications, equivalent replacements, improvements, etc., made without creative efforts, shall be included within the scope of protection of the present disclosure.

What is claimed is:

1. A neutralizing antibody against pulmonary fibrosis, wherein the neutralizing antibody against pulmonary fibrosis consists of a heavy chain and a light chain, wherein the heavy chain comprises a heavy chain variable region comprising a heavy chain complementary-determining region 1 (CDR1) as set forth in SEQ ID NO. 1, a heavy chain complementary-determining region 2 (CDR2) as set forth in SEQ ID NO. 2, and a heavy chain complementary-determining region 3 (CDR3) as set forth in SEQ ID NO. 3; the light chain comprises a light chain variable region comprising a light chain CDR1 as set forth in SEQ ID NO. 4, a light chain CDR2 as set forth in SEQ ID NO. 5, and a light chain CDR3 as set forth in SEQ ID NO. 6.

2. The neutralizing antibody against pulmonary fibrosis according to claim 1, wherein an amino acid sequence of the heavy chain is as set forth in SEQ ID NO. 8, and an amino acid sequence of the light chain is as set forth in SEQ ID NO. 9.

3. A drug, comprising the neutralizing antibody against pulmonary fibrosis according to claim 1, for use in the treatment of pulmonary fibrosis diseases mediated by Gremlin2.

4. The drug according to claim 3, wherein the drug further comprises a pharmaceutically acceptable carrier.


SEQUENCE LISTING (9 sequences)

SEQ ID NO: 1 - protein, 12 aa, synthetic construct
  source 1..12: note = Amino acid sequence of the heavy chain variable region CDR1
VSGDAPPAIGKY

SEQ ID NO: 2 - protein, 7 aa, synthetic construct
  source 1..7: note = amino acid sequence of the heavy chain variable region CDR2
AGGEDGI

SEQ ID NO: 3 - protein, 10 aa, synthetic construct
  source 1..10: note = Amino acid sequence of the heavy chain variable region CDR1
APFGGAAYIE

SEQ ID NO: 4 - protein, 9 aa, synthetic construct
  source 1..9: note = Amino acid sequence of the light chain variable region CDR1
SGGSGADIA

SEQ ID NO: 5 - protein, 6 aa, synthetic construct
  source 1..6: note = Amino acid sequence of the light chain variable region CDR2
NGSLPP

SEQ ID NO: 6 - protein, 8 aa, synthetic construct
  source 1..8: note = Amino acid sequence of the light chain variable region CDR3
AACTRKLT

SEQ ID NO: 7 - protein, 18 aa, synthetic construct
  source 1..18: note = A binding sequence on Gremlin2 to TGFBR1
NSFYIPRHVKKEEDSFQS

SEQ ID NO: 8 - protein, 233 aa, synthetic construct
  source 1..233: note = Amino acid sequence of the heavy chain
EVQLVESGGGLVQPGGSLRLSCAASVSGDAPPAIGKYYMHWVRQAPGKGLEWVGLIAGGEDGITIYDPKFQDRATISADNSKNTAYLQMNSLRAEDTAVYYCARAPFGGAAYIEWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 9 - protein, 210 aa, synthetic construct
  source 1..210: note = Amino acid sequence of the light chain
DIQMTQSPSSLSASVGDRVTITCSGGSGADIAWYQQKPGKAPKVLIYNGSLPPGVPSRFSGSGSGTDYTLTISSLQPEDFATYYCAACTRKLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC